(19) United States
(12) Patent Application Publication
(10) Pub. No.: US 2025/0318946 A1
(43) Pub. Date: Oct. 16, 2025

(54) Methods and Systems for Alternating Pressure Pelvic Compression

(71) Applicant: Matthew Solomon Ross, Arcadia, CA (US)

(72) Inventor: Matthew Solomon Ross, Arcadia, CA (US)

(21) Appl. No.: 19/080,816

(22) Filed: Mar. 15, 2025

Related U.S. Application Data
(60) Provisional application No. 63/571,428, filed on Mar. 28, 2024.

(51) Int. Cl. A61F 5/34; A61F 5/02

(52) U.S. Cl. CPC A61F 5/34; A61F 5/02

(57) ABSTRACT

This disclosure generally relates to a medical system and a method for compressing a human pelvis. This disclosure relates to a pelvic compression system and methods for its use that provide prolonged pelvic compression to a human, avoiding or minimizing the major complication of skin breakdown and/or necrosis. An exemplary pelvic compression system may include a first elongated flexible body, a plurality of elongated flexible chambers, an alternating pressurization system, and a fastening system. This pelvic compression system may further include a second elongated flexible body. This pelvic compression system may further include a pressure-sensing system, or a safety system, or a control system, or a combination of these systems.

### CROSS-REFERENCE TO RELATED APPLICATION

This application is based upon and claims the benefit of U.S. provisional patent application 63/571,428, entitled “Methods and Systems for Alternating Pressure Pelvic Compression,” filed Mar. 28, 2024, attorney docket number MR-24001. The entire content of this provisional patent application is incorporated herein by reference.

### TECHNICAL FIELD

This disclosure generally relates to a medical system and a method for compressing a human pelvis. This disclosure relates to a pelvic compression system and methods for its use that provide prolonged pelvic compression to a human, avoiding or minimizing the major complication of skin breakdown and/or necrosis.

### BACKGROUND OF THE INVENTION

### Description Of The Related Art

Compressive binder devices for severely traumatized pelvic injuries are a well-known treatment in the medical arts. Severe pelvic ring injuries can damage venous and arterial vasculature as well as pelvic and abdominal viscera. Pelvic ring injuries have been shown to have an associated mortality rate of between 10%-50%, depending on the severity of the energy imparted to the pelvis. Rapid exsanguination can occur in severe injuries that damage the pelvic vasculature; aggressive management of this hemorrhage with compression has proven to be the optimal initial treatment.

The pelvic binder or sheet is now the standard of care for the initial treatment of most exsanguinating pelvic ring injuries. Some purposes of the binder or sheet are to (a) Apply a significant amount of constant force to the pelvis to reduce the pelvic volume; (b) splint the bony pelvis to reduce hemorrhage from fractured bone ends and venous disruption; (c) stabilize and maintain the integrity of the pelvis for future definitive surgical treatment; and/or (d) reduce pain.

Conventionally, when deployed uniformly, the pelvic binder may apply pressure on the lateral aspects of the greater trochanters of the hip's proximal femurs to compress the hips from the lateral aspect medially and distribute the compressive force over a wide surface area on the lateral hips. This compressive force and/or its uneven distribution has led to the development of pressure-induced skin and soft tissue breakdown and necrosis. This soft tissue breakdown and necrosis has been shown to significantly increase the morbidity and mortality of the traumatized patient.

Even with the uniform deployment of a pelvic binder or sheet, this medical device can still apply significant compressive force to the pelvis. As a result, even an adequately applied binder or sheet skin can still cause breakdown and necrosis over bony prominences over its long-term use. For this reason, many orthopedic surgeons would elect to remove the pelvic binder after 24 hours of its deployment and surgically apply an external fixator device in its stead to avoid skin breakdown complications.

However, the surgical application of an external fixator device can also cause major possible complications as the patient is often in a critical state of health, and even minor interventions such as anesthesia or mobilization can quickly cause the patient's health status to deteriorate. In many cases, the patient's health is in such a precarious state that longer-term application of the pelvic binder would allow for the stabilization of their overall health status before they go to surgery for either the application of an external fixator or a more definitive surgery.

Traumatic pelvic ring injuries are associated with significant morbidity and mortality (1). Pelvic binders have become the standard of care in the initial treatment and stabilization of most of these life-threatening injuries (2). Pelvic binders are effective due to their ease of application, low cost, and efficacy. These binders provide a quick means of reducing the bony pelvis and stopping internal pelvic bleeding from posterior pelvic venous plexus injuries.

To reduce the bony pelvis and tamponade pelvic bleeding, a significant amount of pressure is usually necessary. This pressure can easily, and most often does, exceed, for example, the 9.8 kilopascal (kPa) gauge pressure limit for tissue perfusion (3). When this exemplary pressure limit is exceeded, the underlying soft tissues become hypoxic and placed at risk for tissue necrosis and pressure ulcers. Studies have shown that pressures twice that of normal capillary arterial pressure for as brief as 2 hours causes irreversible ischemia in animal models, and stage I pressure ulcers can even be seen as early as within 30 minutes of tissue ischemia (4).

Pressure ulcers are serious injuries that can significantly affect a patient's morbidity and even mortality. Patients who have been hospitalized in the intensive care unit for prolonged periods are at great risk of developing these injuries. Medicare data shows the cost to treat hospital-acquired pressure ulcers can range from $20,900 to $151,700 and that each diagnosis adds $43,180 to a hospital stay (8).

While no studies have yet evaluated the amount of time that a pelvic binder can be utilized before tissue necrosis occurs, a consensus among traumatologists is that pelvic binders should be removed before 24 hours of use. After this time limit, the pelvis must undergo a more definitive procedure, namely, internal, or external fixation. A major disadvantage of these treatment options is that many patients who have sustained major pelvic ring injuries have also suffered other serious injuries, including abdominal, chest, and brain trauma (1). These injuries often contribute to patient instability and preclude either internal or external fixation.

The following publications are related art for the background of this disclosure. One-digit or two-digit numbers in the parentheses before each reference correspond to the numbers in parentheses used in the other parts of this disclosure.

(1) Papakostidis C., Giannoudis P.V. Pelvic ring injuries with haemodynamic instability: efficacy of pelvic packing, a systematic review. Injury. 2009;40 (Suppl 4): S53-S61.

(2) Biffl W. L., Smith W R, Moore E E, et al. Evolution of a multidisciplinary clinical pathway for the management of unstable patients with pelvic fractures. Ann Surg 2001; 233:843-50.

(3) Bliss M, Simmi B. When are the seeds of pressure sores sown? BMJ 1999; 319:863-4.

(4) Vohra R, McCollum C. Fortnightly review: pressure sores. BMJ 1994; 309:853-7

(5) Hedrick-Thompson J K. A review of pressure reduction device studies. J Vasc. Nurs. 1992; X: 3-5.

(6) Pieper B. Pressure ulcers: impact, etiology and classification. In: Bryant R, Nix D, eds. Acute and Chronic Wounds: Current Management Concepts. 4th ed. St Louis, MO: Mosby; 2012:123-135.

(7) Jowett A J L, Bowyer G W. Pressure characteristics of pelvic binders. Injury. 2007; 38:118-121.

(8) Meddings J, Reichert H, Rogers MA, et al. Under pressure: financial effect of the Hospital-Acquired conditions Initiative-A Statewide analysis of pressure Ulcer Development and payment. J Am Geriatric Soc 2015; 63:1407-12.

(9) Hyun S, Li X, Vermillion B, et al. Body mass index and pressure ulcers: improved predictability of pressure ulcers in intensive care patients. Am J Crit Care. 2014;23 (6): 494-501. doi: 10.4037/ajcc2014535.

The entire content of each of the above references, including its supplemental content, if available, is incorporated herein by reference.

### SUMMARY OF THE INVENTION

This disclosure generally relates to a medical system and a method for compressing a human pelvis. This disclosure relates to a pelvic compression system and methods for its use that provide prolonged pelvic compression to a human, avoiding or minimizing the major complication of skin breakdown and/or necrosis.

In this disclosure, an exemplary pelvic compression system may include a first elongated flexible body, a plurality of elongated flexible chambers, an alternating pressurization system, and a fastening system. This pelvic compression system may further include a second elongated flexible body. This pelvic compression system may further include a pressure-sensing system, or a safety system, or a control system, or a combination of these systems. The first elongated flexible body may include a first distal end and a second distal end, and an outer surface and an inner surface. The second elongated flexible body may include a first distal end and a second distal end, and an outer surface and an inner surface. The first elongated flexible body's first distal end may be positioned to oppose the first elongated flexible body's second distal end. The plurality of elongated flexible chambers may be positioned along the first elongated flexible body and/or along the second elongated flexible body. This pelvic compression system may further include a supporting member. The fastening system may be configured to allow coupling of the first elongated flexible body's first distal end with the first elongated flexible body's second distal end; and/or to allow coupling of the second elongated flexible body's first distal end with the second elongated flexible body's second distal end.

In this disclosure, the alternating pressurization system may be configured to pressurize at least one elongated flexible chamber to a predetermined pressure and/or depressurize at least one elongated flexible chamber to a predetermined pressure. The predetermined pressure to which at least one elongated flexible chamber is pressurized is hereafter referred to as “predetermined inflation pressure.” The predetermined pressure to which at least one elongated flexible chamber is depressurized is hereafter referred to as “predetermined deflation pressure.” The predetermined inflation pressure may be in a range of 3 kPa gauge pressure to 10 kPa gauge pressure; or 3 kPa gauge pressure to 20 kPa gauge pressure; or 3 kPa gauge pressure to 50 kPa gauge pressure; or 4 kPa gauge pressure to 10 kPa gauge pressure; or 4 kPa gauge pressure to 20 kPa gauge pressure; or 4 kPa gauge pressure to 50 kPa gauge pressure; or-5 kPa gauge pressure to 10 kPa gauge pressure; or 5 kPa gauge pressure to 20 kPa gauge pressure; or 5 kPa gauge pressure to 50 kPa gauge pressure; or 10 kPa gauge pressure to 20 kPa gauge pressure; or 10 kPa gauge pressure to 30 kPa gauge pressure; or 10 kPa gauge pressure to 50 kPa gauge pressure. For example, the predetermined inflation pressure may be at least 12 kPa gauge pressure. The predetermined deflation pressure may be in a range of an atmospheric pressure to a gauge pressure below or equal to 4.5 kPa; or an atmospheric pressure to a gauge pressure below or equal to 4.0 kPa; or an atmospheric pressure to a gauge pressure below or equal to 3.0 kPa; or an atmospheric pressure to a gauge pressure below or equal to 2.0 kPa; or an atmospheric pressure to a gauge pressure below or equal to 1.0 kPa. For example, the predetermined deflation pressure may be an atmospheric pressure.

In this disclosure, the fastening system may be configured to allow coupling of the first elongated flexible body's first distal end with the first elongated flexible body's second distal end; and/or coupling of the second elongated flexible body's first distal end with the second elongated flexible body's second distal end.

In this disclosure, the flexible elongated chambers may be positioned between the first elongated flexible body and the second elongated flexible body.

This disclosure's pelvic compression system may further include a supporting member. An exemplary supporting member may be positioned between the first flexible sheet's inner surface and the second flexible sheet's inner surface.

In this disclosure, the width of the first elongated flexible body and/or second elongated body may be in a range of 4 inches to 10 inches; or 5 inches to 9inches; or 6 inches to 8 inches.

In this disclosure, the pelvic compression system may include at least two elongated flexible chambers, or at least four elongated flexible chambers, or at least six elongated flexible chambers.

In this disclosure, the elongated flexible chambers may be tubular in shape when they are fully inflated. A characteristic diameter of each elongated flexible chamber, when it is fully inflated, may be in a range of 0.5 inches to 3 inches, or 1 inch to 2 inches, or 1 inch to 1.5 inches.

In this disclosure, the alternating pressurization system may be configured to alternately and continuously pressurize at least one elongated flexible chamber to a predetermined pressure and depressurize at least one elongated flexible chamber to a predetermined pressure. The alternating pressurization system may be configured to pressurize at least one elongated flexible chamber to a predetermined pressure and/or depressurize at least one elongated flexible chamber to a predetermined pressure according to a predetermined timing pattern. Each pressurized elongated flexible chamber may be positioned adjacent to a depressurized elongated flexible chamber.

In this disclosure, the elongated flexible chambers may be configured such that, when they are deployed around the pelvis, the elongated flexible chambers can apply substantial opposing forces against the hips of a human. The elongated flexible chambers may be configured such that, when they are deployed around the pelvis, the elongated flexible chambers can apply substantial opposing forces against the hips of a patient and can stabilize the patient's broken pelvic bone(s). The elongated flexible chambers may extend at least partially. The elongated flexible chambers may have sufficient length such that, when they are deployed around the pelvis, the elongated flexible chambers can apply substantial opposing forces against the hips of a human. When the elongated chambers are deployed around the pelvis, the elongated flexible chambers may apply substantial opposing forces against the hips of a human and can stabilize a broken pelvic bone(s).

When the elongated flexible chambers are deployed around the pelvis, they may form a predetermined total width at the hip area such that the elongated flexible chambers can apply substantial opposing forces against the hips of a patient and can stabilize the patient's broken pelvic bone(s). The predetermined total width may be in a range of 0.5 inches to 12 inches, or 1 inch to 10 inches, or 4 inches to 9inches, or 6 inches or 8 inches.

In this disclosure, the fastening system may be configured such that it can temporarily couple the elongated flexible body's first distal end with the elongated flexible body's second distal end such that the elongated flexible chambers can apply substantial opposing forces against the hips of a human after the elongated flexible chambers are deployed around the pelvis.

In this disclosure, the elongated flexible chambers may be deployed around the pelvis for a predetermined time such that damage to tissue is minimized.

After the elongated flexible chambers are deployed around the pelvis, they may be pressurized for no longer than 10 seconds, or 30 seconds, or 60 seconds, or 10 minutes, or 30 minutes, or 60 minutes, or 1 hour, or 2 hours, or 3 hours, or 4 hours, or 5 hours, or 10 hours, or 24 hours.

In this disclosure, the elongated flexible body and the elongated flexible chambers may be flexible enough to conform contours of a human's hips and/or pelvis, when they are deployed around the pelvis.

In this disclosure, the alternating pressurization system can continually maintain the elongated flexible chambers' pressure at a sufficient level to reduce and stabilize a patient's fractured pelvic bone(s).

In this disclosure, the pelvic compression system may be configured to pressurize a first group of the elongated flexible chambers while allowing a second group of the elongated flexible chambers to depressurize and/or pressurize the second group while allowing the first group to depressurize.

In this disclosure, the pelvic compression system may be configured to adjust a predetermined inflation pressure or a predetermined deflation pressure according to the body mass index (BMI) of the human to whom it is applied.

The scope of this disclosure includes any combination of inventive features disclosed in the preceding paragraphs of this section, i.e. “Summary of the Invention.”

These and other components, steps, features, objects, benefits, and advantages will become apparent after reviewing the detailed description of illustrative embodiments, the accompanying drawings, and the claims.

### BRIEF DESCRIPTION OF DRAWINGS

The drawings of this disclosure are illustrative examples. They do not illustrate all examples. Other examples may be used in addition or instead. Details that may be apparent or unnecessary may be omitted to save space or for more effective illustration. Some examples may be practiced with additional components or steps and/or without all the illustrated components or steps. When the same numeral appears in different drawings, it refers to the same or similar components or steps. The drawings are not necessarily to scale.

The patent or application file contains at least one drawing executed in color. The Office will provide copies of this patent or patent application publication with color drawing(s) upon request and payment of the necessary fee.

To further understand the present disclosure's nature, objects, and advantages, the following detailed description should be read in conjunction with the drawings.

In this disclosure, the following reference numbers are used for the features disclosed: a pelvis compression system 100, a first elongated flexible body 110, the first elongated flexible body's outer surface 115, the first elongated flexible body's inner surface 116, a plurality of elongated flexible chambers 120, a strap 130, buckles 140, a fastening device 150, fastener receptacles 160, a pressurization hose(s) 170, an alternating pressurization system 180, a second elongated flexible body 190, a supporting member 200, a pressure sensing system 210, a safety system 220, a control system 230, a pressure sensor 240, a first distal end of the first elongated flexible body 250, a second distal end of the first elongated flexible body 260, and total width of elongated flexible chambers 270. These numerals are labels, not a component or feature of this disclosure's pelvic compression system.

FIG. 1. illustrates an internal, elevational view of an exemplary pelvic compression system of this disclosure.

FIG. 2. illustrates a top view of the exemplary pelvic compression system shown in FIG. 1.

FIG. 3. illustrates an external elevational view of the exemplary pelvic compression system of FIGS. 1 and 2.

FIG. 4. illustrates a cross-sectional view of an exemplary pelvic compression system. In this view, all elongated flexible chambers are in inflated stage. The numerals, 1-6 are shown for demonstrative purposes.

FIG. 5. illustrates a cross-sectional view of an exemplary pelvic compression system of FIGS. 1-3 with its alternating pressurization system, a pressure-sensing system including its (pressure) sensor, a safety system, and a control system.

FIG. 6. shows a photograph of an exemplary pelvic compression system built to carry out experiments on a subject. In this photograph, odd-numbered elongated flexible chambers are inflated, and even-numbered chambers are deflated.

FIG. 7. shows a photograph of an exemplary pelvic compression system of FIG. 6 after it is applied to a subject with a pressure sensing mat (which is green in color) placed between the system and the patient. In this photograph, even-numbered elongated flexible chambers were inflated.

FIG. 8. graphically shows the gauge pressures generated by the application of this disclosure's pelvic compression system as measured by the pressure sensing mat shown in FIG. 7: (A) gauge pressures measured in kPa under even-numbered inflated flexible elongated chambers, and (B) gauge pressures measured in kPa under odd-numbered inflated flexible elongated chambers. Heat map output illustrates pressure intensity (range 0 kPa-30 kPa) at 960 points.

FIG. 9. graphically shows the gauge pressures in kPa generated by the application of (A) the commercial pelvic binder A and (B) the commercial pelvic binder B. Heat map output illustrates pressure intensity (range 0 kPa-30 kPa) at 960 points.

FIG. 10. shows experimental results of the application of this disclosure's pelvic compression system and commercial pelvic binders A and B. Average gauge pressure (kPa) by binder type (mean±SD). The gauge pressure of deflated flexible elongated chambers of this disclosure's pelvic compression system was statistically lower than inflated commercial pelvic binders' pressure (one-way ANOVA F=70.7, DF=3, p<0.05).

FIG. 11. shows experimental results of the application of this disclosure's pelvic compression system and commercial pelvic binders A and B. Peak gauge pressure (kPa) by binder type (mean±SD). The peak gauge pressure of deflated flexible elongated chambers of this disclosure's pelvic compression system was statistically lower than the peak gauge pressure of inflated commercial pelvic binders' average gauge pressure (one-way ANOVA F=29.6, DF=3, p<0.05).

### DETAILED DESCRIPTION

Reference will now be made in detail to presently preferred compositions, embodiments, and methods of the present invention, constituting the best modes of practicing the invention currently known to the inventors. However, it is understood that the disclosed embodiments exemplify the invention that may be embodied in various alternative forms. Therefore, specific details disclosed herein are not to be interpreted as limiting but merely as a representative basis for any aspect of the invention and/or as a representative basis for teaching one skilled in the art to employ the present invention in various ways.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary, percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context indicates otherwise. That is, reference to a component in the singular is intended to include a plurality of components. That is, in this disclosure, the indefinite article “a” and the phrases “one or more” and “at least one” are synonymous and mean “at least one.” Similarly, an element preceded by an “a” or an “an” does not, without further constraints, preclude the existence of additional elements of the identical type.

As used herein, the term “about” means that the amount or value in question may be the specific value designated or some other value in its neighborhood. For example, the term “about” denoting a particular value represents a range within ±5% of the value. For example, the phrase “about 100” indicates a range of 100±5, i.e., the value is in the range of 95 to 105. Generally, when the term “about” is used, it can be expected that similar results or effects according to the invention can be obtained, for example, within a range of ±5% of the indicated value.

As used herein, the term “and/or” means that all or only one of the elements of said group may be present. For example, “A and/or B” shall mean “only A, or only B, or both A and B”. In the case of “only A,” the term also covers the possibility that B is absent, i.e., “only A, but not B.”

It is also to be understood that this invention is not limited to the specific embodiments and methods described below, as specific components and/or conditions may vary. Furthermore, the terminology used herein is used only to illustrate particular embodiments of the present invention and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element outlined in that clause; other elements are not excluded from the claim as a whole.

The phrase “consisting essentially of” limits the scope of a claim to the specified materials or steps, plus those that do not materially affect the essential and novel characteristic(s) of the claimed subject matter.

The phrase “composed of” means “including” or “consisting of.” Typically, this phrase denotes that an object is formed from a material.

Concerning the terms “comprising,” “consisting of,” and “consisting essentially of,” where one of these three terms is used herein, the presently disclosed and claimed subject matter can include the use of either of the other two terms. Similarly, the terms “comprises,” “comprising,” and any other variation in connection with a list of elements in the specification or claims indicate that the list is not exclusive and that other elements may be included. For example, “comprising A” includes “consisting of A” or “consisting essentially of A.”

The term “one or more” means “at least one,” and the term “at least one” means “one or more.” The terms “one or more” and “at least one” include “plurality” as a subset.

Relational terms such as “first” and “second” and the like may be used solely to distinguish one entity or action from another without necessarily requiring or implying any actual relationship or order between them.

The terms “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, ±0.1%, ±0.5%, ±1%, ±2%, ±3%, ±4%, ±5% or ±10% of the value or relative characteristic.

It should also be appreciated that integer ranges explicitly include all intervening integers. For example, the integer range 1-10 explicitly contains 1, 2, 3, 4, 5, 6, 7, 8, 9, and 10. Similarly, the range 1 to 100 contains 1, 2, 3, 4 . . . 10 . . . 20 . . . 50 . . . 76 . . . 83 . . . 97, 98, 99, 100. Similarly, when any range is called for, intervening numbers that are increments of the difference between the upper and lower limits divided by 10 can be taken as alternative upper or lower limits. For example, if the range is 1.1. to 2.1 the following numbers 1.2, 1.3, 1.4, 1.5, 1.6, 1.7, 1.8, 1.9, or 2.0 can be selected as lower or upper limits.

The entire disclosures of U.S. Pat. No. 9,968,475 B2, entitled “Alternating Compression Pelvic Trauma Binder,” to Ross; U.S. patent application Ser. No. 14/856,057, entitled “Alternating Compression Pelvic Trauma Binder,” to Ross; and U.S. patent application Ser. No. 15/837,115, entitled “Alternating Compression Pelvic Trauma Binder,” to Ross are incorporated herein by reference in their entirety.

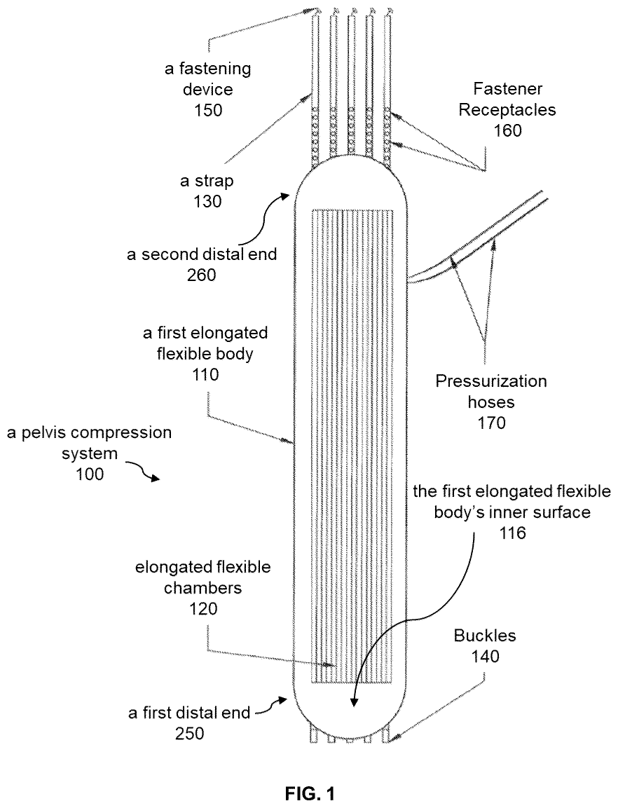

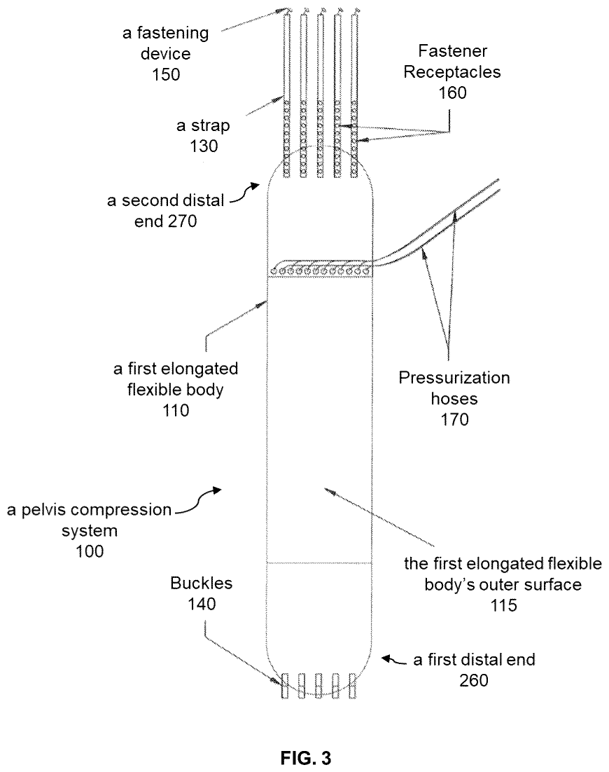

This disclosure relates to a pelvic compression system and methods for its use that provide prolonged pelvic compression to a human, avoiding or minimizing the major complication of skin breakdown and/or necrosis. This human/patient may be healthy or have a health problem. For example, the pelvic compression system(s) of this disclosure may provide support or protection to the healthy human against the possibility of having a pelvic injury. For example, the pelvic compression system(s) of this disclosure may be applied to a severely traumatized patient with a pelvic ring injury to stabilize the patient's health condition, for example, before a further intervention such as surgery. Exemplary pelvic compression systems 100 of this disclosure are shown in FIGS. 1-5.

The present disclosure is generally directed to systems and methods for compressing the pelvis with a plurality of elongated flexible chambers 120 by encircling a human's hip region. In some examples, a pelvic compression system 100 of this disclosure may be applied to a person with a traumatic pelvic injury.

In this disclosure, an exemplary pelvic compression system may include a first elongated flexible body, a plurality of elongated flexible chambers, an alternating pressurization system, and a fastening system. This pelvic compression system may further include a pressure-sensing system, or a safety system, or a control system, or a combination of these systems. This pelvic compression system may further include a second elongated flexible body. The first elongated flexible body may include a first distal end and a second distal end, and an outer surface and an inner surface. The second elongated flexible body may include a first distal end and a second distal end, and an outer surface and an inner surface. The first elongated flexible body's first distal end may be positioned to oppose the first elongated flexible body's second distal end. The plurality of elongated flexible chambers may be positioned along the first elongated flexible body and/or along the second elongated flexible body. This pelvic compression system may further include a supporting member. The fastening system may be configured to allow coupling of the first elongated flexible body's first distal end with the first elongated flexible body's second distal end; and/or to allow coupling of the second elongated flexible body's first distal end with the second elongated flexible body's second distal end.

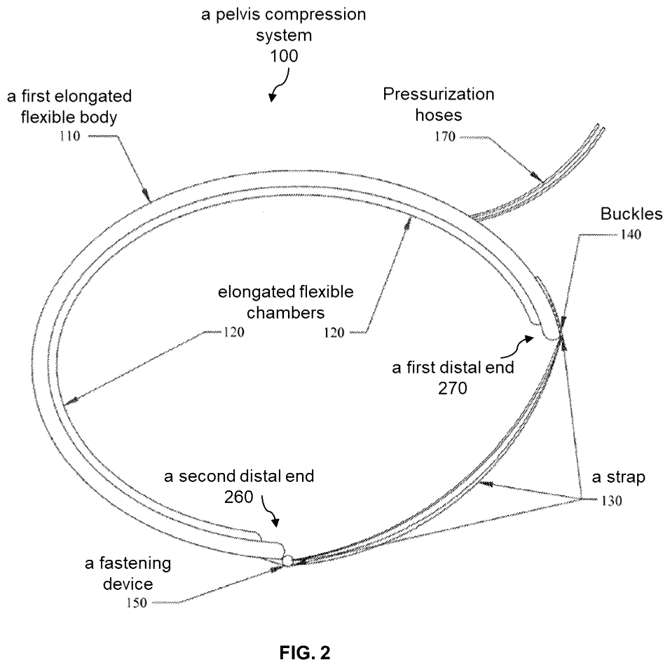

In one example, as shown in FIGS. 1-3, a pelvic compression system 100 may include a first elongated flexible body 110, which may be composed of a flexible material that allows for compression of the bony pelvis. The pelvic compression system 100 may also include a plurality of inflatable elongated flexible chambers 120 that may be arranged in small rings (as shown in FIG. 2) of variable diameters. For example, each of these inflatable elongated flexible chambers 120 may be independently pressurized and/or de-pressurized to a variable pressure setting by using an alternating pressurization system 180.

For example, an alternating pressurization system 180 may pressurize at least one elongated flexible chamber 120 to a predetermined pressure while keeping at least one elongated flexible chamber 120 deflated at any one time. The elongated flexible chambers 120, when deflated, apply no significant pressure to the pelvis. At time intervals (e.g., variable time intervals), at least one deflated elongated flexible chamber 120 may be inflated to a predetermined pressure as at least one inflated elongated flexible chamber 120 then may be deflated, relieving the pressure on the skin and soft tissue under that elongated flexible chamber 120.

For example, this alternating pressurization system 180 may allow for the skin and soft tissue that is being directly compressed by the pressurized elongated flexible chamber(s) 120 to have a set time of depressurization while its adjacent elongated flexible chamber 120 is pressurized and maintain the compressive force on the bony pelvis. This alternating pressurization-depressurization process may allow for blood flow to the soft tissues and skin for a set period before the deflated elongated flexible chamber(s) 120 is compressed again. Because at least one elongated flexible chamber(s) 120 is kept pressurized, the pelvic compression system maintains the compression needed to, for example, inhibit intra-pelvic bleeding while retaining the necessary blood flow to the skin and soft tissues that inhibits the major complication of skin breakdown and necrosis.

In this disclosure, the alternating pressurization system 180 may be configured to pressurize at least one elongated flexible chamber to a predetermined pressure and/or depressurize at least one elongated flexible chamber to a predetermined pressure. The predetermined pressure to which at least one elongated flexible chamber is pressurized is hereafter referred to as “predetermined inflation pressure.” The predetermined pressure to which at least one elongated flexible chamber is depressurized is hereafter referred to as “predetermined deflation pressure.” The predetermined inflation pressure may be in a range of 3 kPa gauge pressure to 10 kPa gauge pressure; or 3 kPa gauge pressure to 20 kPa gauge pressure; or 3 kPa gauge pressure to 50 kPa gauge pressure; or 4 kPa gauge pressure to 10 kPa gauge pressure; or 4 kPa gauge pressure to 20 kPa gauge pressure; or 4 kPa gauge pressure to 50 kPa gauge pressure; or −5 kPa gauge pressure to 10 kPa gauge pressure; or 5 kPa gauge pressure to 20 kPa gauge pressure; or 5 kPa gauge pressure to 50 kPa gauge pressure; or 10 kPa gauge pressure to 20 kPa gauge pressure; or 10 kPa gauge pressure to 30 kPa gauge pressure; or 10 kPa gauge pressure to 50 kPa gauge pressure. For example, the predetermined inflation pressure may be at least 12 kPa gauge pressure. The predetermined deflation pressure may be in a range of an atmospheric pressure to a gauge pressure below or equal to 4.5 kPa; or an atmospheric pressure to a gauge pressure below or equal to 4.0 kPa; or an atmospheric pressure to a gauge pressure below or equal to 3.0 kPa; or an atmospheric pressure to a gauge pressure below or equal to 2.0 kPa; or an atmospheric pressure to a gauge pressure below or equal to 1.0 kPa. For example, the predetermined deflation pressure may be an atmospheric pressure.

The fastening system may be configured to allow coupling of the first elongated flexible body's first distal end with the first elongated flexible body's second distal end; and/or coupling of the second elongated flexible body's first distal end with the second elongated flexible body's second distal end. To achieve such coupling, the fastening system may include a plurality of components such as a strap, a buckle, a fastening device, a fastener receptacle, a belt, a tie, a hook, a loop (such as Velcro®), or a combination of such components. An exemplary fastening system is illustrated in FIGS. 1-3. In this example, the fastening system may include one or many strap apparatuses (or strap) 130 that extend from one end of the pelvic compression system 100 and are looped through buckles 140 on the other end of the pelvic compression system. Such a fastening system may therefore allow for tightening of the pelvic compression system 100 upon itself for compression. The end of the strap 130 may contain a fastening device 150 to affix the end of the strap 130 to itself after the strap 130 has been looped through the buckle 140. The strap 130 may have a plurality of fastener receptacles 160 on its proximal aspect allowing for different mating sites for the fastening device 150. In some examples, the fastening receptacles 160 may be evenly spaced. The fastening receptacles 160 may be placed in variable increments. These different mating sites allow for adjustable tensioning forces to be applied to the pelvis. It is within the scope of the current disclosure to utilize any appropriate fastener 150 as may be known to those of ordinary skill such as, for example: buckles, belts, ties, hook, and loop (such as Velcro®) and the like.

A first elongated flexible body 110 and/or a second elongated flexible body 190 may be manufactured by using a strong, flexible yet non-stretchable material such as neoprene and may have the general shape of a wide belt or pelvic girdle. Such manufacturing may allow for a conforming fit to the individual human/patient yet may not allow for stretching of the material and subsequent loss of compression over time. The outer surface(s) of the first elongated flexible body 110 and/or the second elongated flexible body 190 may be covered with a low friction material to allow for easy maneuverability of the healthy human or the patient while in the supine position. The inner surface(s) of the first elongated flexible body 110 and/or the second elongated flexible body 190 may be covered with a soft cushioning material that allows for even distribution of forces to the patient's body's surface area.

The plurality of elongated flexible chambers 120, when inflated, may gain a cross-sectional shape of a tube with variable diameters. These elongated flexible chambers 120 may be arranged in multiple different orientations, including horizontal, vertical, or at an angle to the first elongated flexible body 110 and/or the second elongated flexible body 190. The elongated flexible chambers 120 may be constructed such that when inflated, these chambers may cause pressure upon the human/patients' hips and subsequently cause pelvic compression. The elongated flexible chambers 120, when deflated, may put lesser or no pressure on the hips when other elongated flexible chambers 120 are inflated and cause compression. In some examples, the elongated flexible chambers 120 may be inflated in an alternating pattern so that every other elongated flexible chamber 120 at any one time may be inflated and causing pressure on the pelvis.

The elongated flexible chambers 120 may be made of an airtight material that is housed directly next to the inner surface(s) of the first elongated flexible body 110 and/or the second elongated flexible body 190. This may allow for the elongated flexible chambers 120 to exert pressure on the hips while not exposing the skin to an abrasive surface area. The cushioning aspect of the first elongated flexible body 110 and/or the second elongated flexible body 190 may shield the body from abrasion and ulceration over time while still allowing for pelvic compression. Each elongated flexible chamber 120 may be coupled to a pressurization hose(s) 170 that allows inflation and/or deflation of the flexible chamber 120. In some examples, the pressurization hoses 170 from the elongated flexible chambers 120 may all exit the first elongated flexible body 110 and/or the second elongated flexible body 190 at one location. The alternating pressurization hoses 170 may be grouped together so that alternate pressurization hoses 170 could be inflated or deflated simultaneously.

In some examples, the pressurization and depressurization of the elongated flexible chambers 120 may be controlled by an external alternating pressurization system 180 that may be programmed to inflate the alternating elongated flexible chambers 120 at set or pre-determined time periods. The pressurization hoses 170 may be of variable lengths and may be coupled to the pressurization device. The alternating pressurization system 180 may be used to set differing levels of compression pressure to the hips.

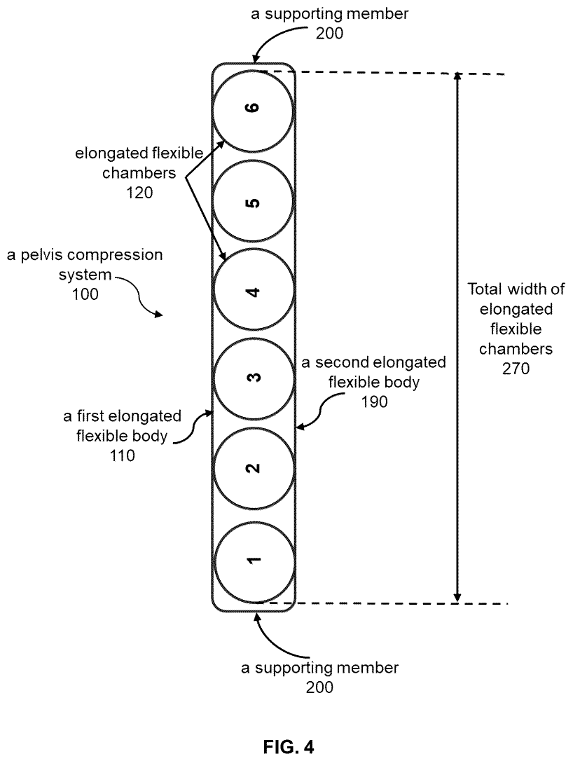

This disclosure's pelvic compression system may further include a second elongated flexible body 190. The second elongated flexible body may include a first distal end, and a second distal end, and an outer surface and an inner surface. The second elongated flexible body's first distal end may be positioned to oppose the second elongated flexible body's second distal end. The first elongated body's inner surface is positioned to face the second elongated body's inner surface. The flexible elongated chambers may be positioned between the first elongated flexible body and the second elongated flexible body, as shown in FIG. 4.

This disclosure's pelvic compression system may further include a supporting member 200. An exemplary supporting member may be positioned between the first flexible sheet's inner surface and the second flexible sheet's inner surface, as shown in FIG. 4.

In this disclosure, the width of the first elongated flexible body and/or second elongated body may be in a range of 4 inches to 10 inches; or 5 inches to 9 inches; or 6 inches to 8 inches.

In this disclosure, the pelvic compression system may include at least two elongated flexible chambers, or at least four elongated flexible chambers, or at least six elongated flexible chambers.

In this disclosure, the elongated flexible chambers may be tubular in shape when they are fully inflated. A characteristic diameter of each elongated flexible chamber, when it is fully inflated, may be in a range of 0.5 inches to 3 inches, or 1 inch to 2 inches, or 1 inch to 1.5 inches.

In this disclosure, the alternating pressurization system may be configured to alternately and continuously pressurize at least one elongated flexible chamber to a predetermined pressure and depressurize at least one elongated flexible chamber to a predetermined pressure. The alternating pressurization system may be configured to pressurize at least one elongated flexible chamber to a predetermined pressure and/or depressurize at least one elongated flexible chamber to a predetermined pressure according to a predetermined timing pattern. Each pressurized elongated flexible chamber may be positioned adjacent to a depressurized elongated flexible chamber.

In this disclosure, the elongated flexible chambers may be configured such that, when they are deployed around the pelvis, the elongated flexible chambers can apply substantial opposing forces against the hips of a human. The elongated flexible chambers may be configured such that, when they are deployed around the pelvis, the elongated flexible chambers can apply substantial opposing forces against the hips of a patient and can stabilize the patient's broken pelvic bone(s). The elongated flexible chambers may extend at least partially. The elongated flexible chambers may have sufficient length such that, when they are deployed around the pelvis, the elongated flexible chambers can apply substantial opposing forces against the hips of a human. When the elongated chambers are deployed around the pelvis, the elongated flexible chambers may apply substantial opposing forces against the hips of a human and can stabilize a broken pelvic bone(s).

When the elongated flexible chambers are deployed around the pelvis, they may form a predetermined total width at the hip area such that the elongated flexible chambers can apply substantial opposing forces against the hips of a patient and can stabilize the patient's broken pelvic bone(s). The predetermined total width may be in a range of 0.5 inches to 12 inches, or 1 inch to 10 inches, or 4 inches to 9 inches, or 6 inches to 8 inches. The total width of the elongated flexible chambers is shown by way of example in FIG. 4.

In this disclosure, the fastening system may be configured such that it can temporarily couple the elongated flexible body's first distal end with the elongated flexible body's second distal end such that the elongated flexible chambers can apply substantial opposing forces against the hips of a human after the elongated flexible chambers are deployed around the pelvis.

In this disclosure, the elongated flexible chambers may be deployed around the pelvis for a predetermined time to minimize tissue damage. After the elongated flexible chambers are deployed around the pelvis, they may be pressurized for no longer than 10 seconds, or 30 seconds, or 60 seconds, or 10 minutes, or 30 minutes, or 60 minutes, or 1 hour, or 2 hours, or 3 hours, or 4 hours, or 5 hours, or 10 hours, or 24 hours.

In this disclosure, the elongated flexible body and the elongated flexible chambers may be flexible enough to conform to the contours of a human's hips and/or pelvis when deployed around the pelvis.

In this disclosure, the alternating pressurization system can continually maintain the elongated flexible chambers' pressure sufficiently to reduce and stabilize a patient's fractured pelvic bone(s).

In this disclosure, the pelvic compression system may be configured to pressurize a first group of the elongated flexible chambers while allowing a second group of the elongated flexible chambers to depressurize, and/or pressurize the second group while allowing the first group to depressurize.

In this disclosure, the pelvic compression system may be configured to adjust a predetermined inflation pressure or a predetermined deflation pressure according to the body mass index (BMI) of the human to whom it is applied.

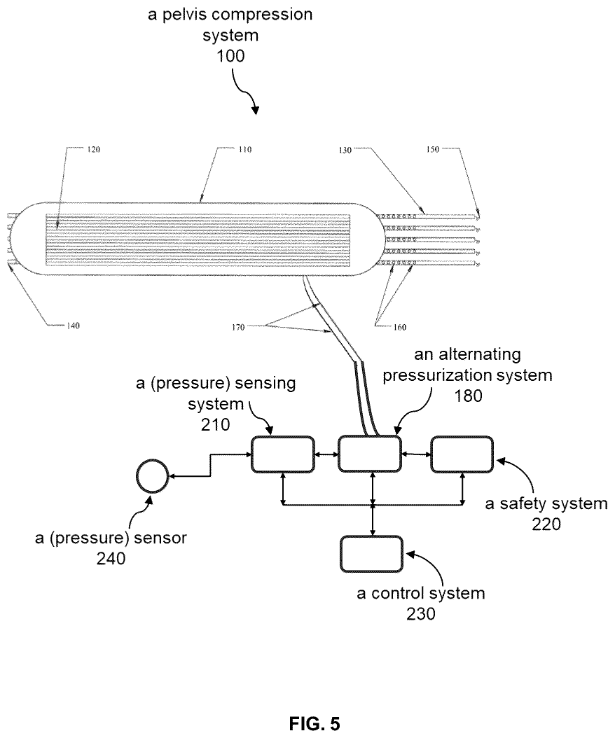

In this disclosure, the pelvic compression system may further include a (pressure) sensing system 210, or a safety system 220, or a control system 230, or a combination of these systems, as shown in FIG. 5. The sensing system 210 may comprise a (pressure) sensor 240.

In this disclosure, the sensing system may be configured to sense a health parameter(s) of a human to whom the pelvic compression system is applied. The sensing system may comprise a sensor to measure these health parameters. Based on measured health parameters, the predetermined inflation pressure and/or the predetermined deflation pressure of the elongated flexible chambers may be adjusted; for example, all flexible chambers may be fully inflated to a maximum predetermined pressure or fully deflated to an atmospheric pressure. The health parameter may, for example, be the blood pressure of a human to whom the pelvic compression system is applied. For example, a sensing system of this disclosure may include a blood pressure measurement device (monitor). An exemplary blood pressure monitor may include an inflatable cuff that can compress and then release a human artery under the cuff in a controlled manner, a pressure gauge, a display, and a controlling system of the blood pressure monitor.

In this disclosure, the safety system may be any system that is configured to provide safety to a human to whom the pelvic compression system is applied. For example, the safety system may deflate the elongated flexible chamber(s) if a pressure of an elongated chamber exceeds a predetermined maximum pressure. For example, the safety system may inflate the elongated flexible chamber(s) to a predetermined maximum pressure if the human gets destabilized.

In this disclosure, the control system may be a system configured to receive information from the sensing system, the alternating pressurization system, the safety system, or a combination of these systems; and/or control information from the sensing system, the alternating pressurization system, the safety system, or a combination of these systems. The control system may use the information it receives from the sensing system, the alternating pressurization system, the safety system, or a combination of these systems to control said systems. For example, if the sensing system measures blood pressure that is indicative of the poor health of a human, the control system may use this measurement to fully inflate or fully deflate or adjust the pressures of the flexible elongated chambers. For example, the control system may control the elongated flexible chambers' predetermined inflation/deflation pressures at predetermined values for predetermined periods. For example, the control system may fully inflate or fully deflate elongated flexible chambers after a predetermined maximum period for the application of the pelvic compression is exceeded. For example, based on a body mass index (BMI) of a human to whom the pelvic compression system is applied, the control system may adjust the flexible elongated chambers' predetermined inflation and deflation pressures. The control system may include a processor and a software installed in a processor component such as memory.

The scope of this disclosure includes any combination of inventive features disclosed in the preceding paragraphs of this section, Detailed Description.

EXAMPLE 1. An Exemplary Pelvic Compression System and Measurement of Pressures Developed on the Bony Prominences of the Pelvis When This System was Applied.

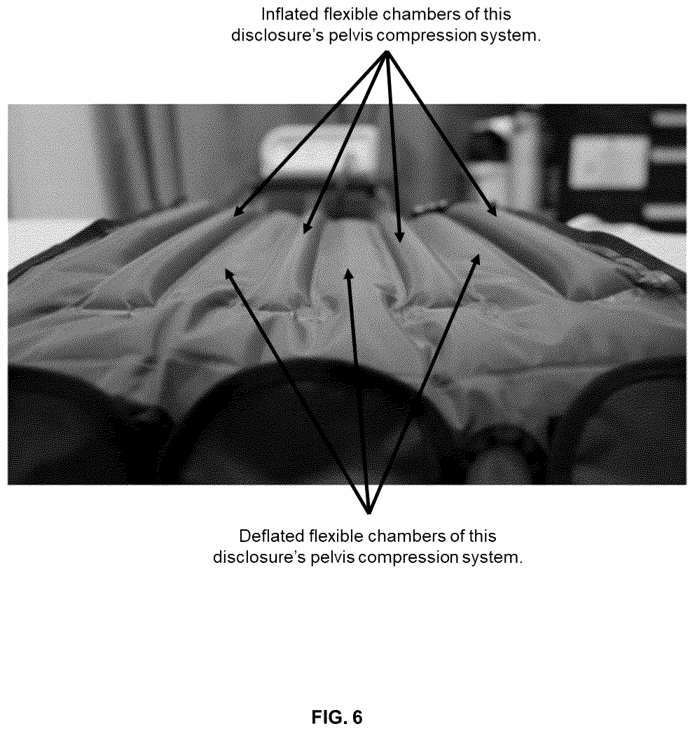

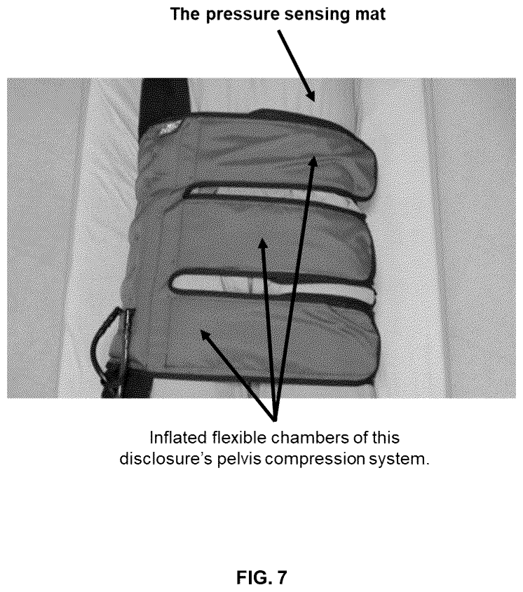

This example relates to an exemplary pelvic compression system manufactured to measure pressures this system generated on the pelvis after it was applied to a subject. FIG. 6 shows a photograph of this exemplary pelvic compression system of this disclosure with inflated and deflated flexible chambers. FIG. 7 shows a photograph of this exemplary pelvic compression system after it was applied to a subject.

Pressures developed at the bony prominences of the pelvis after the pelvic compression system of this disclosure was applied were determined. These measured gauge pressures were compared to those determined by the application of two commercially available pelvic binders labeled A and B.

The pelvic compression system and the commercially available pelvic binders were applied to four healthy individuals (subjects). These subjects underwent sequential testing of this disclosure's pelvic compression system and the two commercial pelvic binders. The possibility of pressure ulcer development was likely affected by the body mass index (BMI) of these subjects, which was in the range of 23.7 to 27.5, with an average of 25.66.

The subjects, dressed in standard hospital regulation top and bottom scrubs, lay down in a hospital bed in a supine position. A thin and flexible pressure sensing mat (BodiTrak BT1510, Vista Medical Ltd., Winnipeg) was then applied over the right greater trochanter. We then sequentially applied this disclosure's pelvic compression system and the two commercial pelvic binders, each time allowing about 10 minutes for the pressure sensing mat to stabilize. Each commercial pelvic binder was applied according to the manufacturer's recommendations.

The gauge pressure (kPa) exerted by the pelvic binders over the greater trochanter was recorded using software from the BodiTrak system (Biocompression Systems, Inc., New Jersey), which generated a graphed digital display of the measured data. The BodiTrak system has 1024 (32×32 array) ½″×½″ sensors contained within a thin flexible mat. Repeat testing of the pressure sensing mat showed reproducible results.

This disclosure's pelvic compression system was applied in the same fashion as the commercial pelvic binders. After application, it was connected to the compression system using its two compression hoses, one for the even-numbered chambers (2-4-6-8) and one for the odd-numbered chambers (1-3-5-7). After allowing for stabilization of the pressure sensing mat system, the alternating pressurization system was turned on. The alternating pressurization system was initially programmed to inflate all even-numbered chambers and odd-numbered chambers. After one minute of compression from both even-numbered chambers and odd-numbered chambers, the even-numbered chambers were deflated for about one-minute intervals, leaving the odd-numbered chambers inflated to compress the pelvis. After that one-minute interval, the even-numbered chambers were re-inflated, and the odd-numbered chambers were deflated for another one-minute interval. This sequence of alternatingly inflating-deflating of the groups of elongated flexible chambers ran for a total of six one-minute intervals. The gauge pressures developed under both inflated and deflated elongated flexible chambers were recorded during each inflation-deflation interval.

The gauge pressures were recorded as both peak pressure and average pressure in kPa. The average gauge pressure for each subject was found by taking 10 pressure readings from the area over the greater trochanter and averaging those readings for each subject. The peak gauge pressure was found by finding the highest pressure reading over the greater trochanter from each measurement. This was done for this disclosure's pelvic compression system and the two commercial pelvic binders.

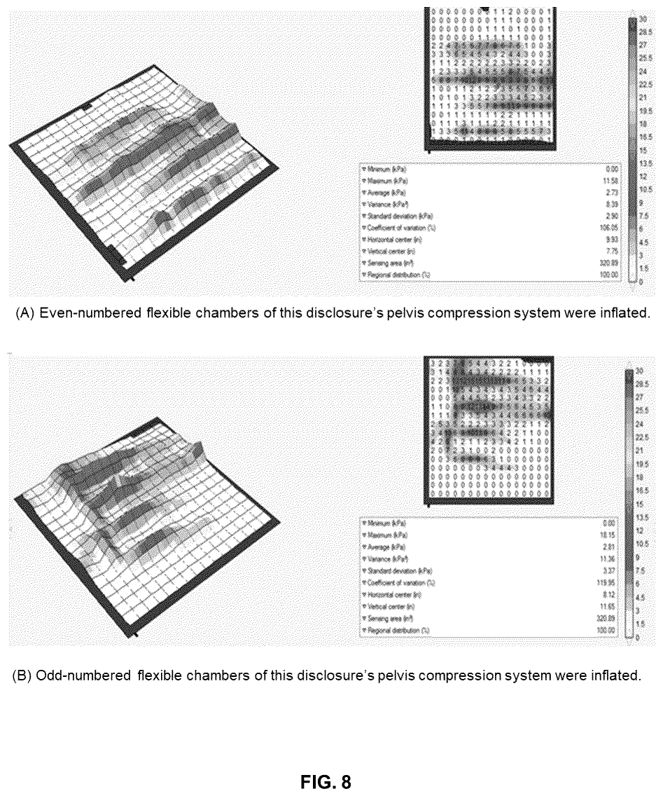

It was measured that the inflated flexible chambers of this disclosure's pelvic compression system applied an average gauge pressure of 11.43 kPa and a peak gauge pressure of 13.97 kPa; and the deflated flexible chambers of this disclosure's pelvic compression system applied an average gauge pressure of 2.74kPa and a peak gauge pressure of 4.31 kPa. See FIG. 8.

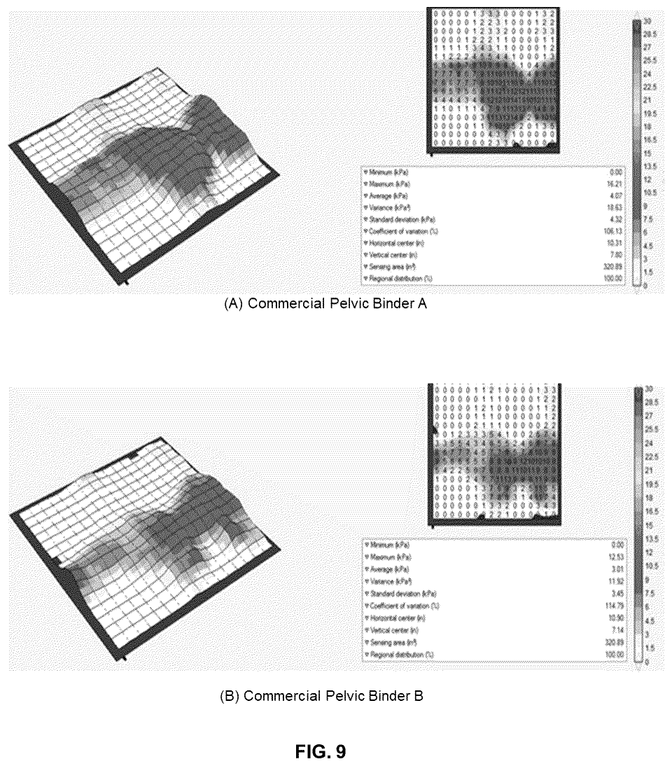

The commercial pelvic binder A applied an average gauge pressure of 9.9 kPa and a peak gauge pressure of 13.28 kPa, as shown in FIG. 9(A). The commercial binder B applied an average gauge pressure of 12.12 kPa and a peak gauge pressure of 17.21 kPa, as shown in FIG. 9(B).

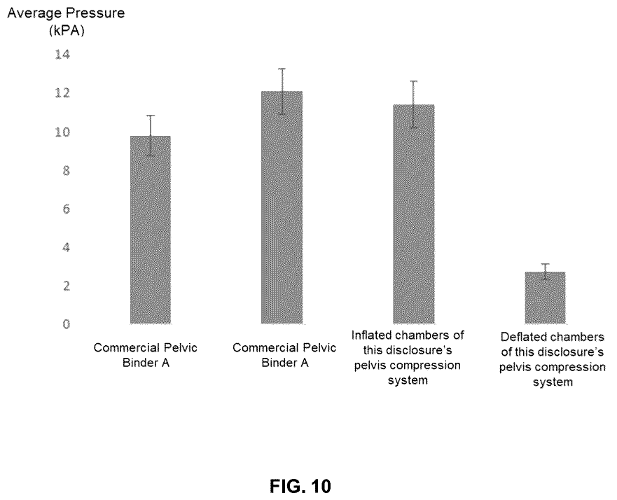

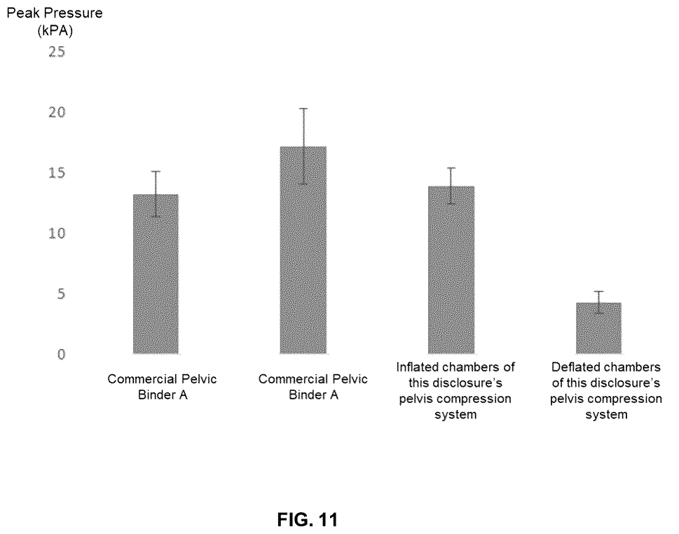

We determined that the pressures developed by the commercial pelvic binders over the bony prominences were all greater than the recommended pressure, about 5 kPa, for avoiding the development of pressure ulcers, as shown in FIGS. 10-11.

When applied, this disclosure's pelvic compression system generated the same pressures as the commercial binders in the inflated elongated flexible chambers, which can reduce the pelvis and tamponade pelvic bleeding, as shown in FIGS. 10-11. However, the deflated elongated flexible chambers produced significantly less pressure over the bony prominences, which could allow for normal tissue perfusion.

These results indicated that severely injured patients who have sustained pelvic ring fractures and are treated by using this disclosure's pelvic compression system could wear the said system for a prolonged period and forgo risky interventional or surgical procedures until these patients are stabilized. That is, this disclosure's pelvic compression system can allow for soft tissue perfusion under its elongated flexible chambers. By alternately inflating and deflating these chambers, a user of this disclosure's pelvic compression system can minimize or even prevent the development of pressure ulcers and tissue necrosis.

This disclosure's pelvic compression system generated the same necessary pressures as the commercial pelvic binders to reduce the pelvis and tamponade pelvic bleeding, as the alternating chambers in this binder allowed for continuous compression while minimizing the risk of tissue necrosis.

The commercial pelvic binders are used to stabilize patients with severe pelvic ring injuries initially. In many cases, such pelvic binders may be removed safely after the initial stabilization period but before the risk of developing pressure ulcers begins. However, some, for example, severely injured patients may require a prolonged application of the pelvic binder in the intensive care unit before they can safely be transported to the operating room for internal or external fixation.

For such patients, a continual treatment modality to stabilize their pelvis with minimal risk for soft tissue necrosis would be greatly advantageous. A pelvic compression system that could be utilized for days, possibly weeks, without causing pressure ulcers and still allow for medical stabilization of the patient before going to the operating room could negate the need to perform high-risk surgical procedures on unstable patients. A pelvic compression system, which could be applied for elongated periods, may potentially alleviate the need to perform an external fixation procedure on the pelvis and allow for the stabilization of the patient before performing the definitive internal fixation procedure to treat the pelvic injury. As the experimental results demonstrated, this disclosure's pelvic compression system can be used for such treatment purposes.

The scope of this disclosure includes any combination of inventive features disclosed in the preceding paragraphs of this section, i.e., “Detailed Description.”

Unless otherwise stated, all measurements, values, ratings, positions, magnitudes, sizes, and other specifications outlined in this specification, including in the following claims, are approximate, not exact. They are intended to have a reasonable range consistent with the functions to which they relate and with what is customary in the art to which they pertain.

As will be understood by one skilled in the art, for any purposes, such as providing a written description, all ranges disclosed herein also encompass any possible sub-ranges and combinations of sub-ranges thereof. Any listed range can be easily recognized as sufficiently describing and enabling the same range to be broken down into at least equal halves, thirds, quarters, fifths, tenths, etc. As a non-limiting example, each range discussed herein can be readily broken down into a lower third, middle third, upper third, etc. As will also be understood by one skilled in the art of all languages, such as “up to,” “at least,” “greater than,” “less than,” and the like include the number recited and refer to ranges which can be subsequently broken down into sub-ranges as discussed herein. Finally, as will be understood by one skilled in the art, a range includes each member. Thus, for example, a group with 1-3 articles refers to groups with 1, 2, or 3 articles. Similarly, a group having 1-5 articles refers to groups having 1, 2, 3, 4, or 5 articles, and so forth.

While various aspects and embodiments have been disclosed herein, others will be apparent to those skilled in the art. The various aspects and embodiments disclosed herein are for illustration purposes and are not intended to be limiting.

All references cited herein, including but not limited to published and unpublished applications, patents, and literature references, are incorporated herein by reference for the subject matter referenced and, in their entirety, are made a part of this specification. To the extent publications and patents or patent applications incorporated by reference contradict the disclosure contained in the specification, the specification is intended to supersede and take precedence over any such contradictory material.

Concerning the use of substantially any plural and/or singular terms herein, those with skill in the art can translate from the plural to the singular and/or from the singular to the plural as appropriate to the context and/or application. The various singular/plural permutations may be expressly set forth herein for clarity.

The phrase “means for” when used in a claim is intended to and should be interpreted to embrace the corresponding structures and materials that have been described and their equivalents. Similarly, when used in a claim, the phrase “step for” is intended to and should be interpreted to embrace the corresponding acts described and their equivalents. The absence of these phrases from a claim means that the claim is not intended to and should not be interpreted as limited to these corresponding structures, materials, or acts, or to their equivalents.

In at least some of the previously described embodiments, one or more elements used in an embodiment can interchangeably be used in another embodiment unless such a replacement is not technically feasible. It will be appreciated by those skilled in the art that various other omissions, additions, and modifications may be made to the methods and structures described herein without departing from the scope of the claimed subject matter. All such modifications and changes are intended to fall within the scope of the disclosed subject matter.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). Those within the art will further understand that if a specific number of an introduced claim recitation is intended, such an intent will be explicitly recited in the claim, and, in the absence of such recitation, no such intent is present. For example, as an aid to understanding, the following appended claims may use the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also described in terms of any individual member or subgroup of members of the Markush group.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been said or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the detailed description are grouped in various embodiments to streamline the disclosure. This method of disclosure should not be interpreted as requiring claimed embodiments to require more features than are expressly recited in each claim. Instead, as the following claims reflect, inventive subject matter lies in less than all features of a single disclosed embodiment. Thus, the following claims are hereby incorporated into the detailed description, with each claim standing on its own as separately claimed subject matter.

The scope of this disclosure includes any combination of inventive features disclosed in the preceding paragraphs of this section or the paragraphs of the following sections.

While exemplary embodiments are described above, these embodiments are not intended to represent all possible forms of the invention. Instead, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. A pelvic compression system, comprising:
a first elongated flexible body;
a plurality of elongated flexible chambers;
an alternating pressurization system; and
a fastening system;
wherein:
the first elongated flexible body comprises a first distal end and a second distal end, and an outer surface and an inner surface;
the first elongated flexible body's first distal end is positioned to oppose the first elongated flexible body's second distal end;
the plurality of elongated flexible chambers are positioned along the first elongated flexible body;
the alternating pressurization system is configured to pressurize at least one elongated flexible chamber to a predetermined inflation pressure and/or depressurize at least one elongated flexible chamber to a predetermined deflation pressure;
the predetermined inflation pressure is in a range of a gauge pressure above atmospheric pressure to 50 kPa;
the predetermined deflation pressure is in a range of an atmospheric pressure to a gauge pressure below or equal to 10 kPa; and
the fastening system is configured to allow coupling of the first elongated flexible body's first distal end with the first elongated flexible body's second distal end.

2. The pelvic compression system of claim 1, wherein the pelvic compression system further comprises a second elongated flexible body; wherein the second elongated flexible body comprises a first distal end and a second distal end, and an outer surface and an inner surface; wherein the second elongated flexible body's first distal end is positioned to oppose the second elongated flexible body's second distal end; and wherein the first elongated body's inner surface is positioned to face the second elongated body's inner surface.

3. The pelvic compression system of claim 1, wherein the predetermined inflation pressure is in a range of 3 kPa gauge pressure to 10 kPa gauge pressure; or 3 kPa gauge pressure to 20 kPa gauge pressure; or 3 kPa gauge pressure to 50 kPa gauge pressure; or 4 kPa gauge pressure to 10 kPa gauge pressure; or 4 kPa gauge pressure to 20 kPa gauge pressure; or 4 kPa gauge pressure to 50 kPa gauge pressure; or 5 kPa gauge pressure to 10 kPa gauge pressure; or 5 kPa gauge pressure to 20 kPa gauge pressure; or 5 kPa gauge pressure to 50 kPa gauge pressure; or 10 kPa gauge pressure to 20 kPa gauge pressure; or 10 kPa gauge pressure to 30 kPa gauge pressure; or 10 kPa gauge pressure to 50 kPa gauge pressure.

4. The pelvic compression system of claim 1; wherein the predetermined deflation pressure is in a range of an atmospheric pressure to a gauge pressure below or equal to 4.5 kPa; or an atmospheric pressure to a gauge pressure below or equal to 4.0 kPa; or an atmospheric pressure to a gauge pressure below or equal to 3.0 kPa; or an atmospheric pressure to a gauge pressure below or equal to 2.0 kPa; or an atmospheric pressure to a gauge pressure below or equal to 1.0 kPa.

5. The pelvic compression system of claim 1, wherein the pelvic compression system further comprises a pressure sensing system; and wherein the pressure sensing system is configured to regulate the predetermined inflation pressure and/or the predetermined deflation pressure.

6. The pelvic compression system of claim 5, wherein the pressure sensing system comprises a blood pressure measurement device.

7. The pelvic compression system of claim 1, wherein the pelvic compression system further comprises a safety system; and wherein the safety system is configured to inflate all chambers to a predetermined pressure.

8. The pelvic compression system of claim 1, wherein the flexible elongated chambers extend at least partially or substantially from the elongated flexible body's first distal end to the elongated flexible body's second distal end.

9. The pelvic compression system of claim 1, wherein the flexible elongated chambers are positioned between the first elongated flexible body and the second elongated flexible body.

10. The pelvic compression system of claim 1, wherein the elongated flexible chambers are positioned between the first elongated flexible body's inner surface and the second elongated flexible body's inner surface.

11. The pelvic compression system of claim 1, wherein the pelvic compression system further comprises a supporting member; and wherein the supporting member is positioned between the first flexible sheet's inner surface and the second flexible sheet's inner surface.

12. The pelvic compression system of claim 1, wherein the alternating pressurization system is configured to pressurize at least one elongated flexible chamber to a predetermined pressure and/or depressurize at least one elongated flexible chamber to a predetermined pressure according to a predetermined timing pattern.

13. The pelvic compression system of claim 1, wherein the elongated flexible chambers are configured such that, when they are deployed around the pelvis, the elongated flexible chambers can apply substantial opposing forces against the hips of a human.

14. The pelvic compression system of claim 1, wherein the elongated flexible chambers are configured such that, when they are deployed around the pelvis, the elongated flexible chambers can apply substantial opposing forces against the hips of a patient and can stabilize the patient's broken pelvic bone(s).

15. The pelvic compression system of claim 1, wherein, when the elongated flexible chambers are deployed around the pelvis, they form a predetermined total width at the hip area such that the elongated flexible chambers can apply substantial opposing forces against the hips of a patient and can stabilize the patient's broken pelvic bone(s).

16. The pelvic compression system of claim 1, wherein the elongated flexible chambers are deployed around the pelvis for a predetermined time such that damage to tissue is minimized.

17. The pelvic compression system of claim 1, wherein, after the elongated flexible chambers are deployed around the pelvis, they are pressurized to the predetermined inflation pressure or the predetermined deflation pressure for no longer than 10 seconds, or 30 seconds, or 60 seconds, or 10 minutes, or 30 minutes, or 60 minutes, or 1 hour, or 2 hours, or 3 hours, or 4 hours, or 5 hours, or 10 hours, or 24 hours.

18. The pelvic compression system of claim 1, wherein the alternating pressurization system can continually maintain the elongated flexible chambers' pressure at a sufficient level to reduce and stabilize a patient's fractured pelvic bone(s).

19. The pelvic compression system of claim 1, wherein the pelvic compression system is configured to adjust a predetermined inflation pressure or a predetermined deflation pressure according to a body mass index (BMI) of a human to whom the pelvic compression system is applied.

20. The pelvic compression system of claim 1, wherein:
the predetermined inflation pressure is in a range of 3 kPa gauge pressure to 10 kPa gauge pressure; or 3 kPa gauge pressure to 20 kPa gauge pressure; or 3 kPa gauge pressure to 50 kPa gauge pressure; or 4 kPa gauge pressure to 10 kPa gauge pressure; or 4 kPa gauge pressure to 20 kPa gauge pressure; or 4 kPa gauge pressure to 50 kPa gauge pressure; or 5 kPa gauge pressure to 10 kPa gauge pressure; or 5 kPa gauge pressure to 20 kPa gauge pressure; or 5 kPa gauge pressure to 50 kPa gauge pressure; or 10 kPa gauge pressure to 20 kPa gauge pressure; or 10 kPa gauge pressure to 30 kPa gauge pressure; or 10kPa gauge pressure to 50 kPa gauge pressure; and
the predetermined deflation pressure is in a range of an atmospheric pressure to a gauge pressure below or equal to 4.5 kPa; or an atmospheric pressure to a gauge pressure below or equal to 4.0 kPa; or an atmospheric pressure to a gauge pressure below or equal to 3.0 kPa; or an atmospheric pressure to a gauge pressure below or equal to 2.0 kPa; or an atmospheric pressure to a gauge pressure below or equal to 1.0 kPa; and
the pelvic compression system further comprises a pressure sensing system; and wherein the pressure sensing system is configured to regulate the predetermined inflation pressure and/or the predetermined deflation pressure; and
the pelvic compression system further comprises a safety system; and wherein the safety system is configured to inflate all chambers to a predetermined pressure; and
after the elongated flexible chambers are deployed around the pelvis, they are pressurized to the predetermined inflation pressure or the predetermined deflation pressure for no longer than 10 seconds, or 30 seconds, or 60 seconds, or 10 minutes, or 30 minutes, or 60 minutes, or 1 hour, or 2 hours, or 3 hours, or 4 hours, or 5 hours, or 10 hours, or 24 hours.
